(19) United States
(12) Patent Application Publication
(10) Pub. No.: US 2025/0049339 A1
(43) Pub. Date: Feb. 13, 2025

(54) LASER SPECKLE CONTRAST IMAGING SYSTEM AND LASER SPECKLE CONTRAST IMAGING METHOD THEREOF

(71) Applicant: National Health Research Institutes, Miaoli County (TW)

(72) Inventors: Lun-De Liao, Miaoli County (TW); Chia-Hui Tsao, Miaoli County (TW)

(73) Assignee: National Health Research Institutes, Miaoli County (TW)

(21) Appl. No.: 18/719,840

(22) PCT Filed: Dec. 16, 2021
(86) PCT No.: PCT/US2021/063925

§ 371 Date: Jun. 14, 2024

(51) Int. Cl. A61B 5/026; A61B 5/00; A61B 5/1455; G02B 27/48

(52) U.S. Cl. CPC A61B 5/0261; A61B 5/0077; A61B 5/1455; G02B 27/48

(57) ABSTRACT

A laser speckle contrast imaging system includes a laser light source, configured to emit a laser beam on a subject; a plurality of visible light sources, configured to respectively emit a visible light beam on the subject; an image capturing module, configured to obtain a first image corresponding to the laser beam emitting toward the subject and a plurality of second images corresponding to the plurality of visible light beams emitting toward the subject; and an image processor, coupled to the image capturing module, configured to process the first image with a laser speckle stacking algorithm to determine a variation of a cortical spreading depression (CSD) waveform according to at least a speckle pattern determined in the first image.

### BACKGROUND OF THE INVENTION

### 1. Field of the Invention

The present invention relates to a laser speckle contrast imaging system and a laser speckle contrast imaging method thereof, and more particularly, to a laser speckle contrast imaging system and a laser speckle contrast imaging method thereof capable of improving a calculation efficiency of cortical spreading depression.

### 2. Description of the Prior Art

Clinically, the non-invasive blood microcirculation inspection is a way to find out possible diseases, and is utilized for assisting the medical care. In addition, smart technologies are adopted in related medical imaging facilities for monitoring peripheral blood circulation, which reflects a body condition, e.g. bruise inspection, acute or chronic wound healing, hypertension, hyperlipidemia, arteriosclerosis or cardiovascular diseases.

There are two conventional medical technologies for monitoring peripheral blood circulation: (1) Laser Doppler velocimetry (LDF) technology, which may be implemented with a small laser fiber optic probe and would not affect other facilities. However, the facilities with LDF technology have challenges, such as (a) single point measurement, (b) vibration noises generated when operated by hands, (c) poor accuracy.

(2) Near-infrared spectroscopy (NIR) technology, which improves the disadvantages of the LDF technology. However, the facilities with NIR technology have challenges, such as (a) lack of resolution and shooting area, (b) unadjustable machine height, (3) manual adjustment to a camera and non-adjustable region of interest (ROI).

Therefore, improvements are necessary to the conventional technique.

### SUMMARY OF THE INVENTION

In light of this, the present invention provides a laser speckle contrast imaging system and a laser speckle contrast imaging method thereof which improves a calculation efficiency of cortical spreading depression.

An embodiment of the present invention discloses a laser speckle contrast imaging system, comprises a laser light source, configured to emit a laser beam on a subject; a plurality of visible light sources, configured to respectively emit a visible light beam on the subject; an image capturing module, configured to obtain a first image corresponding to the laser beam emitting toward the subject and a plurality of second images corresponding to the plurality of visible light beams emitting toward the subject; and an image processor, coupled to the image capturing module, configured to process the first image with a laser speckle stacking algorithm to determine a variation of a cortical spreading depression (CSD) waveform according to at least a speckle pattern determined in the first image.

Another embodiment of the present invention discloses a laser speckle contrast imaging method, for a laser speckle contrast imaging system, wherein the laser speckle contrast imaging system includes a laser light source, an image capturing module and an image processor, and the laser speckle contrast imaging method comprises emitting, by the laser light source, a laser beam on a subject and respectively emitting, by a plurality of visible light sources, a visible light beam on the subject; obtaining, by the image capturing module, a first image corresponding to the laser beam emitting toward the subject and a plurality of second images corresponding to the plurality of visible light beams emitting toward the subject; and processing, by an image processor, the first image with a laser speckle stacking algorithm to determine a variation of a cortical spreading depression (CSD) waveform according to at least a speckle pattern determined in the first image.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a schematic diagram of a laser speckle contrast imaging (LSCI) system according to an embodiment of the present invention.

FIG. 2 is a schematic diagram of the LSCI system for performing an imaging process on a subject according to an embodiment of the present invention.

FIG. 3 is a schematic diagram of a laser speckle contrast imaging (LSCI) system according to another embodiment of the present invention.

FIG. 4 is a schematic diagram of a laser speckle image according to an embodiment of the present invention.

FIG. 5 is a schematic diagram of cortical spreading depression (CSD) waveforms of speckles on the laser speckle image according to an embodiment of the present invention.

FIG. 6 is a schematic diagram of a laser speckle contrast imaging method according to an embodiment of the present invention.

### DETAILED DESCRIPTION

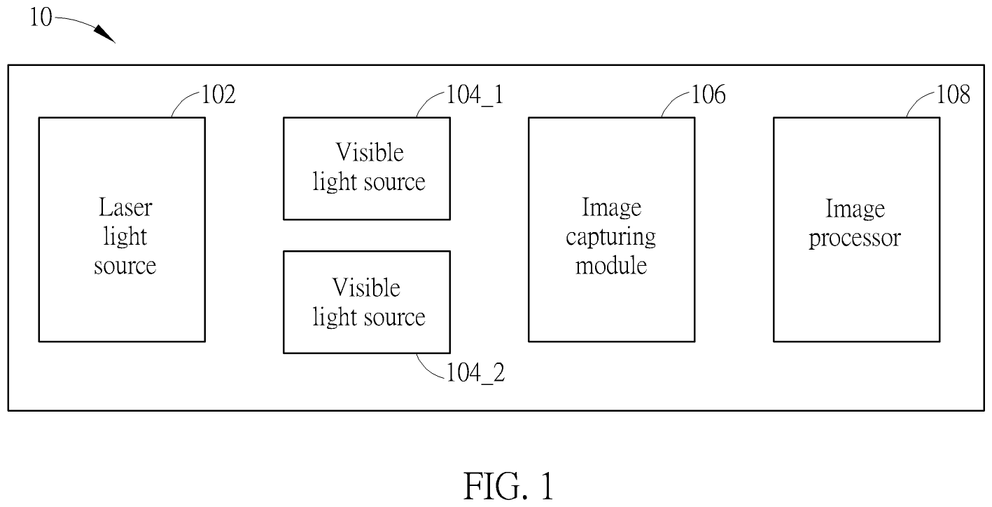

Please refer to FIG. 1, which is a schematic diagram of a laser speckle contrast imaging (LSCI) system 10 according to an embodiment of the present invention. The LSCI system 10 may be utilized for monitoring the blood microcirculation, which includes a laser light source 102, a plurality of visible light sources 104_1, 104_2, an image capturing module 106 and an image processor 108.

The laser light source 102 is configured to emit a laser beam on a subject, e.g. a laser beam with wavelength of 820 nm, and the subject may be a tissue surface. The visible light sources 104_1, 104_2 are configured to respectively emit visible light beams on the subject. In an embodiment, the visible light sources 104_1, 104_2 may respectively be visible lights with wavelengths of 532 nm and 660 nm. The image capturing module 106 is configured to obtain a first image corresponding to the laser beam emitting toward the subject and a plurality of second images corresponding to the visible light beams emitting toward the subject. The image processor 108 is coupled to the image capturing module 106 and is configured to process the first image with a laser speckle stacking algorithm to determine a variation of a cortical spreading depression (CSD) waveform according to at least a speckle pattern determined in the first image. In an embodiment, the image processor 108 may be a device with computing ability. Therefore, the LSCI system 10 according to an embodiment of the present invention may improve calculation efficiency of CSD.

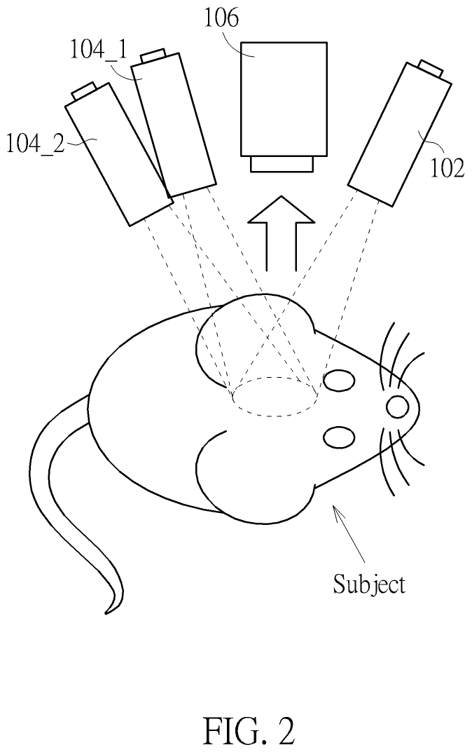

In detail, please refer to FIG. 2, which is a schematic diagram of the LSCI system 10 for performing the imaging process on the subject according to an embodiment of the present invention. The subject, i.e. a mouse brain in the figure, is illuminated by the laser light source 102 and the visible light sources 104_1, 104_2 at the same time, and the LSCI system 10 is utilized for capturing the first image and the second image in a specific time period, such that the laser speckle patterns of the first image and the second image are collected. In addition, compared to conventional technology, which only acquires laser speckle data samples, an emitting frequency of the laser light source 102 according to an embodiment of the present invention is stimulated by a digital control method to obtain the laser speckle data with higher resolution of capillaries in the images, wherein the digital control method is to digitally control energy and frames of the laser beam.

Notably, the subject illustrated in FIG. 2 is not limited to the mouse brain, skin wounds of animal or human are applicable to the present invention.

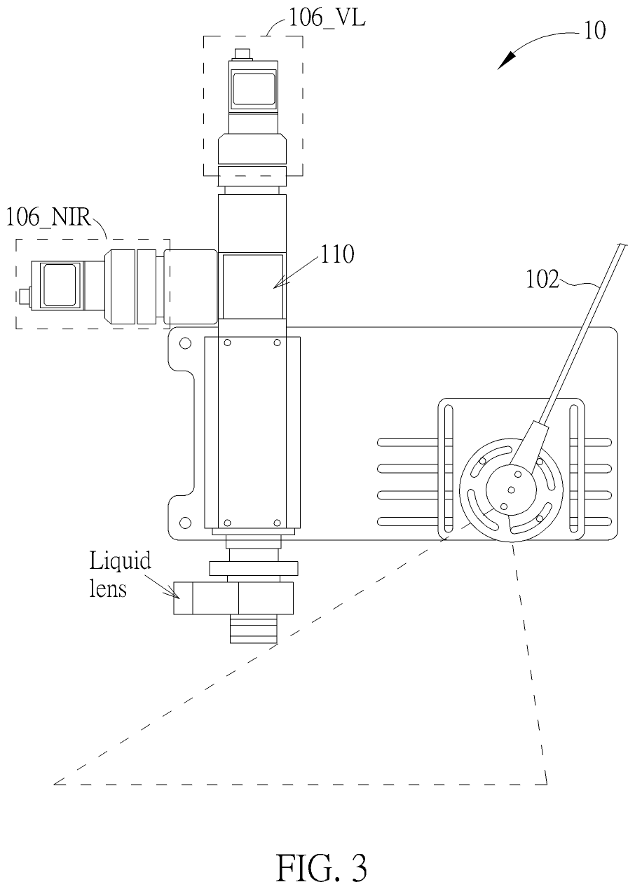

FIG. 3 is a schematic diagram of a laser speckle contrast imaging (LSCI) system 10 according to another embodiment of the present invention. As shown in FIG. 3, the LSCI system 10 further includes a beam splitter 110, which is configured to split the laser beam of the illuminated subject and the visible light beams of the illuminated subject. As such, the beam splitter 110 may split the laser light beam and the visible light beam illuminated on the subject.

In addition, the image capturing module 106 includes a near infrared (NIR) camera 106_NIR and a visible light camera 106_VL. The NIR camera 106_NIR is configured to generate the first image of the subject illuminated by the laser beam from the laser light source 102. The visible light camera 106_VL is configured to generate the second images of the subject illuminated by the visible light beams from the visible light sources 104_1, 104_2. With multiple light sources illuminated on the subject, the light absorbances of the tissue surface of the subject under different light sources are collected by the image capturing module 106, such that the image processor 108 according to an embodiment of the present invention may determine the speckle pattern of the first image and the second images with an adaptive algorithm. The adaptive algorithm is related to different light absorbances under different light sources of the subject, such that the resolution of the laser speckle contrast imaging is increased.

The image processor may determine a blood flow velocity distribution of the first image and the second images with a variation of a colormap of the first image and the second images. In detail, the blood flow information under around 2 nm of the tissue surface may be obtained, which is illuminated by the laser light source 102, and the blood oxygen of the second images may be determined, which is illuminated by the visible light sources 104_1, 104_2.

Further, after the blood flow velocity distribution of the first image and the second images is determined, the image processor 108 is configured to determine a region of interest (ROI) in the first image and the second images. In an embodiment, the image processor 108 may automatically select different ROI for imaging, and the selected ROI may be utilized for measurements of blood flow and blood oxygen.

Note that, the LSCI system 10 may further include a liquid lens to rapidly perform auto-focus to improve efficiency of the examination.

In addition, the laser light beam may consequently monitor an area of the tissue surface, i.e. blood microcirculation, of the subject up to around 40 square meters (cm²), and a resolution of single capillary obtained may be up to 3 um per pixel.

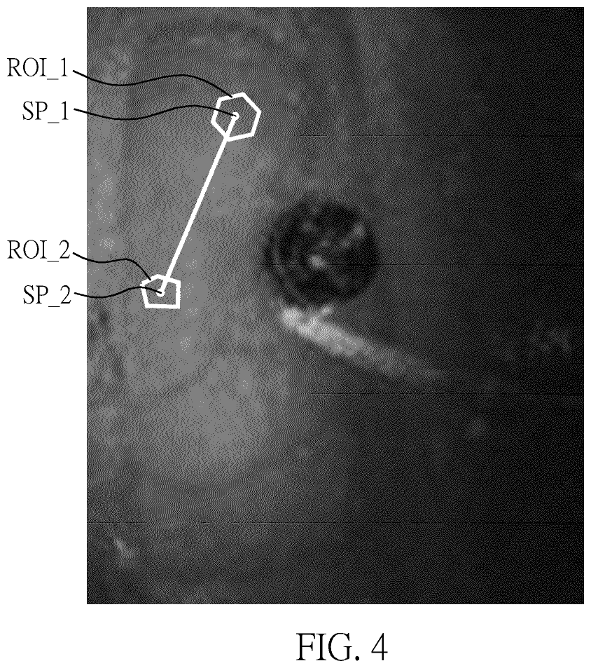

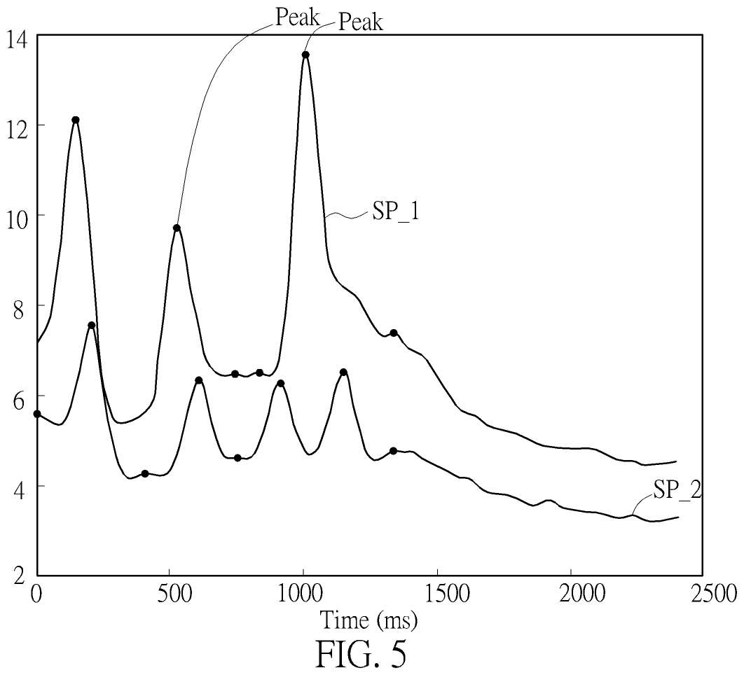

In this regard, the laser speckle stacking algorithm, which is utilized for determining the variation of the CSD waveform according to the speckle patterns in the first image. Please refer to FIGS. 4 and 5. FIG. 4 is a schematic diagram of a laser speckle image according to an embodiment of the present invention and FIG. 5 is a schematic diagram of the CSD waveforms of speckles on the first image according to an embodiment of the present invention.

As shown in FIG. 4, coordinates of a first speckle pattern SP_1 are determined within a region of interest ROI_1, coordinates of a second speckle pattern SP_2 are determined within a region of interest ROI_2, wherein the region of interest ROI_1 is determined at a time t_1 and the region of interest ROI_2 is determined at a time t_2. FIG. 5 illustrates the CSD waveforms of the first speckle pattern SP_1 and the second speckle pattern SP_2 versus time (ms). In addition, CSD waveform peaks of the first speckle pattern SP_1 and the second speckle pattern SP_2 are labeled.

The image processor 108 may determine a CSD velocity V_CSD of the ROI according to a distance D_12 between the region of interest ROI_1 and the second speckle pattern SP_2 of the first image obtained at different times and a time difference of a peak of the CSD waveform corresponding to the first speckle pattern SP_1 and the second speckle pattern SP_2. In other words, the CSD velocity V_CSD may be summarized as a formula (1):

$$V\_CSD = D\_ 12 / ( t 2 - t 1 ) \qquad ( 1 )$$

For example, the distance D_12 between the first speckle pattern SP_1 and the second speckle pattern SP_2 may be measured in real-time during a surgery, such that the CSD velocity may be determined according to the time difference corresponding to the peak of the CSD waveform, which improves the calculation efficiency of CSD velocity.

In addition, the image processor 108 may determine a direction of the CSD of the ROI according to a location of each of speckle patterns SP_1, SP_2 of the first image obtained at different times.

In this way, the LSCI system 10 according to an embodiment of the present invention may be utilized for monitoring and quantizing the data of the peripheral blood flow, e.g. brain and wound region of animal, in the medical field.

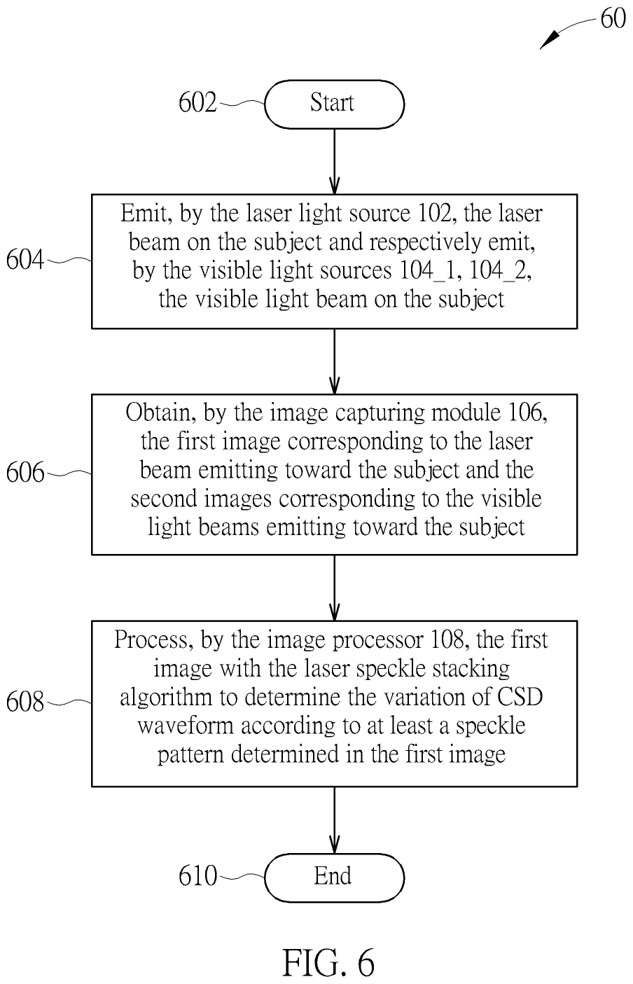

Furthermore, an operation method of the LSCI system 10 may be summarized as a laser speckle contrast imaging method 60, as shown in FIG. 6. The laser speckle contrast imaging method 60 includes the following steps:
Step 602: Start;
Step 604: Emit, by the laser light source 102, the laser beam on the subject and respectively emit, by the visible light sources 104_1, 104_2, the visible light beam on the subject;
Step 606: Obtain, by the image capturing module 106, the first image corresponding to the laser beam emitting toward the subject and the second images corresponding to the visible light beams emitting toward the subject;
Step 608: Process, by the image processor 108, the first image with the laser speckle stacking algorithm to determine the variation of CSD waveform according to at least a speckle pattern determined in the first image;
Step 610: End.

Regarding operations of laser speckle contrast imaging method 60, please refer to the above mentioned embodiments of the LSCI system 10, which is not narrated herein again for brevity.

In summary, the present invention provides a laser speckle contrast imaging system and a laser speckle contrast imaging method thereof which improves a calculation efficiency of cortical spreading depression, improves the resolution of the laser speckle imaging by light beams with multiple waveforms, and improves the efficiency of determining the CSD velocity.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A laser speckle contrast imaging system, comprising:
a laser light source, configured to emit a laser beam on a subject;
a plurality of visible light sources, configured to respectively emit a visible light beam on the subject;
an image capturing module, configured to obtain a first image corresponding to the laser beam emitting toward the subject and a plurality of second images corresponding to the plurality of visible light beams emitting toward the subject; and
an image processor, coupled to the image capturing module, configured to process the first image with a laser speckle stacking algorithm to determine a variation of a cortical spreading depression (CSD) waveform according to at least a speckle pattern determined in the first image.

2. The laser speckle contrast imaging system of claim 1, wherein an emitting frequency of the laser light source is stimulated by a digital control method.

3. The laser speckle contrast imaging system of claim 1, wherein the image processor is configured to determine the speckle pattern of the first image and the plurality of second images with an adaptive algorithm.

4. The laser speckle contrast imaging system of claim 3, wherein the adaptive algorithm is related to different light absorbances under different light sources of the subject.

5. The laser speckle contrast imaging system of claim 1, wherein the image processor is configured to determine a blood flow velocity distribution of the plurality of second images with a variation of a colormap of the plurality of second images.

6. The laser speckle contrast imaging system of claim 1, wherein the image processor is configured to determine a region of interest (ROI) in the first image and the plurality of second images.

7. The laser speckle contrast imaging system of claim 6, wherein the image processor is configured to determine a CSD velocity of the ROI according to a distance between a first speckle pattern and a second speckle pattern of the first image obtained at different times and a time difference of a peak of the CSD waveform corresponding to the first speckle pattern and the second speckle pattern.

8. The laser speckle contrast imaging system of claim 6, wherein the image processor is configured to determine a direction of the CSD of the ROI according to a location of each of multiple speckle patterns of the first image obtained at different times.

9. The laser speckle contrast imaging system of claim 1, wherein the image capturing module comprises:
a near infrared (NIR) camera, configured to generate the first image of the subject illuminated by the laser beam; and
a visible light camera, configured to generate the plurality of second images of the subject illuminated by the plurality of visible light beams.

10. The laser speckle contrast imaging system of claim 1, further comprising:
a beam splitter, coupled to the image capturing module, configured to split the laser beam of the illuminated subject and the plurality of visible light beams of the illuminated subject.

11. A laser speckle contrast imaging method, for a laser speckle contrast imaging system, wherein the laser speckle contrast imaging system includes a laser light source, an image capturing module and an image processor, and the laser speckle contrast imaging method comprising:
emitting, by the laser light source, a laser beam on a subject and respectively emitting, by a plurality of visible light sources, a visible light beam on the subject;
obtaining, by the image capturing module, a first image corresponding to the laser beam emitting toward the subject and a plurality of second images corresponding to the plurality of visible light beams emitting toward the subject; and
processing, by an image processor, the first image with a laser speckle stacking algorithm to determine a variation of a cortical spreading depression (CSD) waveform according to at least a speckle pattern determined in the first image.

12. The laser speckle contrast imaging method of claim 11, wherein an emitting frequency of the laser light source is stimulated by a digital control method.

13. The laser speckle contrast imaging method tem of claim 11, further comprising:
determining, by the image processor, the speckle pattern of the first image and the plurality of second images with an adaptive algorithm.

14. The laser speckle contrast imaging method of claim 13, wherein the adaptive algorithm is related to different light absorbances under different light sources of the subject.

15. The laser speckle contrast imaging method of claim 11, further comprising:
determining, by the image processor, a blood flow velocity distribution of the ROI of the plurality of second images with a variation of a colormap of the plurality of second images.

16. The laser speckle contrast imaging method of claim 11, further comprising:
determining, by the image processor, a region of interest (ROI) in the first image and the plurality of second images.

17. The laser speckle contrast imaging method of claim 16, further comprising:
determining, by the image processor, a CSD velocity of the ROI according to a distance between a first speckle pattern and a second speckle pattern of the first image obtained at different times and a time difference of a peak of the CSD waveform corresponding to the first speckle pattern and the second speckle pattern.

18. The laser speckle contrast imaging method of claim 16, further comprising:
determining, by the image processor, a direction of the CSD of the ROI according to a location of each of multiple speckle patterns of the first image obtained at different times.

19. The laser speckle contrast imaging method of claim 11, wherein the image capturing module comprises:
a near infrared (NIR) camera, configured to generate the first image of the subject illuminated by the laser beam; and
a visible light camera, configured to generate the plurality of second images of the subject illuminated by the plurality of visible light beams.

20. The laser speckle contrast imaging method of claim 11, wherein the laser speckle contrast imaging system comprises:
a beam splitter, coupled to the image capturing module, configured to split the laser beam of the illuminated subject and the plurality of visible light beams of the illuminated subject.
